(19) United States
(12) Patent Application Publication
(10) Pub. No.: US 2008/0249580 A1
(43) Pub. Date: Oct. 9, 2008

(54) Methods and Instruments of Reducing a Fracture

(75) Inventors: David L. Evans, Bartlett, TN (US); Gene Edward Austin, Bartlett, TN (US); Nathaniel Kelley Grusin, Germantown, TN (US); Sied W. Janna, Memphis, TN (US); James K. Rains, Cordova, TN (US); Mark S. Gosney, Cordova, TN (US); Nicholas S. Ritchey, Collierville, TN (US); Henry B. Faber, Memphis, TN (US); Kamrin R. Landrem, Memphis, TN (US)

Correspondence Address: JOEL R. PETROW;SMITH & NEPHEW, INC., 1450 BROOKS ROAD, MEMPHIS, TN, 38116, US

(73) Assignee: SMITH & NEPHEW, INC., Memphis, TN (US)

(21) Appl. No.: 12/088,541

(22) PCT Filed: Sep. 28, 2006
(86) PCT No.: PCT/US06/37588

§ 371 Date: Mar. 28, 2008

Related U.S. Application Data
(60) Provisional application No. 60/721,367, filed on Sep. 28, 2005.
(60) Provisional application No. 60/721,369, filed on Sep. 28, 2005.

(51) Int. Cl. A61F 5/04; A61F 5/00

(52) U.S. Cl. 606/86 R; 606/105; 606/237

(57) ABSTRACT

An instrument for reduction of a bone fracture is disclosed. The instrument includes an orthopaedic surgical implant (112), an implant member (190, 200, 270), and a driving member (180, 210, 260). The implant member (190, 200, 270) has a bone engagement portion (195, 203, 275) and a driven portion (197, 208, 278). The driving member (180, 210, 260) cooperates with the driven portion (197, 208, 278) to move the implant member (190, 200, 270) and reduce the fracture. Also disclosed is a sliding compression orthopaedic implant (300). The implant (300) comprises a first implant member (310), said first implant member (310) having a transverse hole (311); and a second implant member (312) connected to said transverse hole (311), said second implant member (312) having a shank (314), and said shank (314) having a bone engagement portion (316) at a first end portion (318) and a sliding compression member (320) at a second end portion (322).

### CROSS-REFERENCE TO RELATED APPLICATIONS

This application claims the benefit of U.S. Provisional Application No. 60/721,367, filed Sep. 28, 2005 and U.S. Provisional Application No. 60/721,369, filed Sep. 28, 2005. The disclosure of each application is incorporated by reference in its entirety.

### STATEMENT REGARDING FEDERALLY SPONSORED RESEARCH OF DEVELOPMENT

Not Applicable.

### APPENDIX

Not Applicable.

### BACKGROUND OF THE INVENTION

1. Field of the Invention

This invention relates generally to orthopaedic instrumentation and, more particularly, to internal fixation of a fracture.

2. Related Art

It is often desired to achieve reduction of a fracture prior to insertion of an implant into the fracture location. It is difficult to obtain an accurate reduction after an implant has been inserted. Further, it is difficult to accurately reduce a fracture with an implant.

In addition to the problems of fracture reduction, there have also been problems in treating proximal femoral fractures and femoral shaft fractures. There are a variety of devices used to treat femoral fractures. Fractures of the femoral neck have been successfully treated with a variety of compression screw assemblies that include a compression plate having a barrel member, a lag screw and a compressing screw. For unstable subtrochanteric fractures, the extreme loads have frequently caused implants, such as hip compression screw plates, to fail. Proximal femoral fractures and femoral shaft fractures have been treated with help of intramedullary rods that are inserted into the canal of the femur to immobilize the femur parts involved in fractures. A single angled cross-nail or locking screw is inserted through the femur and the proximal end of the intramedullary rod. In cases of severe comminution of the femoral shaft, existing interlocking nails have not provided adequate strength.

Traditional lag screws as well as compression plates with barrel members have been used in the past. These devices may not always offer sufficient strength when they experience high loads. Existing interlocking nails cannot provide enough strength in a highly comminuted fracture.

There remains a need in the art for methods and devices to provide surgeons with the techniques and instrumentation necessary to achieve an accurate and anatomical reduction prior to the insertion of a more permanent implant. Further, there remains a need in the art for more effective treatment of proximal femoral fractures and femoral shaft fractures.

### SUMMARY OF THE INVENTION

It is in view of the above problems that the present invention was developed.

The invention is an orthopaedic instrument that allows for preliminary reduction of a fracture prior to fixation of the bone fragments. The orthopaedic instrument engages a bone fragment and allows a surgeon to manipulate the fragment for accurate reduction. In some embodiments, the orthopaedic instrument includes an orthopaedic surgical implant, such as an intramedullary nail or extramedullary plate.

The invention also relates to devices for treating femoral fractures. Femoral fractures are usually treated with the help of an intramedullary nail that is inserted into the canal of the femur to fixate the portions of the femur that are fractured. The invention provides a treatment for fracture fixation that allows the fracture to be reduced and loaded. The invention is a sliding compression orthopaedic implant. Sliding compression permits variations in loading of the fracture without compromising the anatomical reduction that is desired.

The method and instrumentation described herein provide the advantage of enabling a surgeon to reduce a fracture correctly before putting an implant into the body. Prior art implants attempt to achieve reduction with an implantable device that is placed into the body prior to reducing the fracture and often leads to a fracture reduction that is not stable or anatomically correct. These could lead to non-unions or mal-unions. The methods and instrumentation described herein also offers the advantage of being minimally invasive. Several methods also reduce the need for multiple holes to be drilled thereby avoiding the occurrence of stress risers.

Thus, in furtherance of the above goals and advantages, the present invention is, briefly, an instrument for reduction of a fracture of a bone. The instrument includes an orthopaedic surgical implant, an implant member, and a driving member. The orthopaedic surgical implant, such as an intramedullary nail or extramedullary plate, has a longitudinally extending bore and a transverse hole. The implant member is associated with or connected to the transverse hole. The implant member has a shank with a bone engagement portion at a first end portion and a driven portion at a second end portion. The driving member is in driving engagement with the implant member. The driving member has a shaft with a driving arm at a third end portion, the shaft is sized to fit within the longitudinally extending bore of the orthopaedic surgical implant, and the driving arm is selectively engaged with the driven portion of the implant member. When the driving member is rotated, the implant member moves in order to reduce the fracture.

Further, the invention is, briefly, a sliding compression orthopaedic implant. The implant includes a first implant member and a second implant member. The first implant member, such as an intramedullary nail or extramedullary plate, has a transverse hole, and the second implant member is associated with or connected to the transverse hole. The second implant member has a shank, and the shank has a bone engagement portion at a first end portion and a sliding compression member at a second end portion. The sliding compression orthopaedic implant maintains the reduction of the fracture but allows for dynamic loading to aid in fracture healing.

Further features, aspects, and advantages of the present invention, as well as the structure and operation of various embodiments of the present invention, are described in detail below with reference to the accompanying drawings.

### BRIEF DESCRIPTION OF THE DRAWINGS

The accompanying drawings, which are incorporated in and form a part of the specification, illustrate the embodiments of the present invention and together with the description, serve to explain the principles of the invention. In the drawings:

FIG. 1 is a sectional side view of an instrument for reduction of a bone fracture in a first embodiment;

FIG. 2 is a detailed view of the embodiment shown in FIG. 1;

FIG. 3 is a sectional side view of an instrument for reduction of a bone fracture in a second embodiment;

FIG. 4 is a detailed view of the embodiment shown in FIG. 3;

FIG. 5 is a sectional side view of an instrument for reduction of a bone fracture in a third embodiment;

FIG. 6 is a detailed view of the embodiment shown in FIG. 5;

FIG. 7 is a sectional side view of an instrument for reduction of a bone fracture in a fourth embodiment;

FIG. 8 is a sectional side view of an instrument for reduction of a bone fracture in a fifth embodiment;

FIG. 9 is a sectional side view of the fifth instrument in an alternative configuration;

FIG. 10 is a sectional side view of an instrument for reduction of a bone fracture in a sixth embodiment;

FIG. 11 is a sectional side view of an alternative configuration of the sixth instrument;

FIG. 12 is a sectional side view of an instrument for reduction of a bone fracture in a seventh embodiment in a first configuration;

FIG. 13 is a detailed view of the embodiment shown in FIG. 12;

FIG. 14 is a sectional side view of the seventh embodiment in a second configuration;

FIG. 15 is a sectional side view of an instrument for reduction of a bone fracture in an eighth embodiment;

FIG. 16 is a sectional side view of an instrument for reduction of a bone fracture in a ninth embodiment;

FIG. 17 is a sectional side view of an instrument for reduction of a bone fracture in a tenth embodiment;

FIG. 18 is a sectional side view of a sliding compression orthopaedic implant in a first embodiment;

FIG. 19 is a detailed view of the embodiment shown in FIG. 18;

FIG. 20 is a sectional side view of a sliding compression orthopaedic implant in a second embodiment;

FIG. 21 is a sectional side view of a sliding compression orthopaedic implant in a third embodiment;

FIG. 22 is a detailed view of the embodiment shown in FIG. 21;

FIG. 23 is a sectional side view of a sliding compression orthopaedic implant in a fourth embodiment;

FIG. 24 is a sectional side view of a sliding compression orthopaedic implant in a fifth embodiment;

FIG. 25 is a detailed view of the embodiment shown in FIG. 24;

FIG. 26 is a sectional side view of an intramedullary nail in a first embodiment;

FIG. 27 is a detailed view of the intramedullary nail shown in FIG. 28;

FIG. 28 is a sectional side view of an intramedullary nail in a second embodiment;

FIG. 29 is a detailed view of the intramedullary nail shown in FIG. 26;

FIG. 30 is a sectional side view of an intramedullary nail in a third embodiment;

FIG. 31 is a detailed view of the intramedullary nail shown in FIG. 30;

FIG. 32 is a sectional side view of an intramedullary nail in a fourth embodiment;

FIG. 33 is a detailed view of the embodiment shown in FIG. 32;

FIG. 34 is a sectional side view of a sliding compression orthopaedic implant in a sixth embodiment; and

FIG. 35 is a sectional side view of an instrument for reduction of a bone fracture in an eleventh embodiment.

### DETAILED DESCRIPTION OF THE EMBODIMENTS

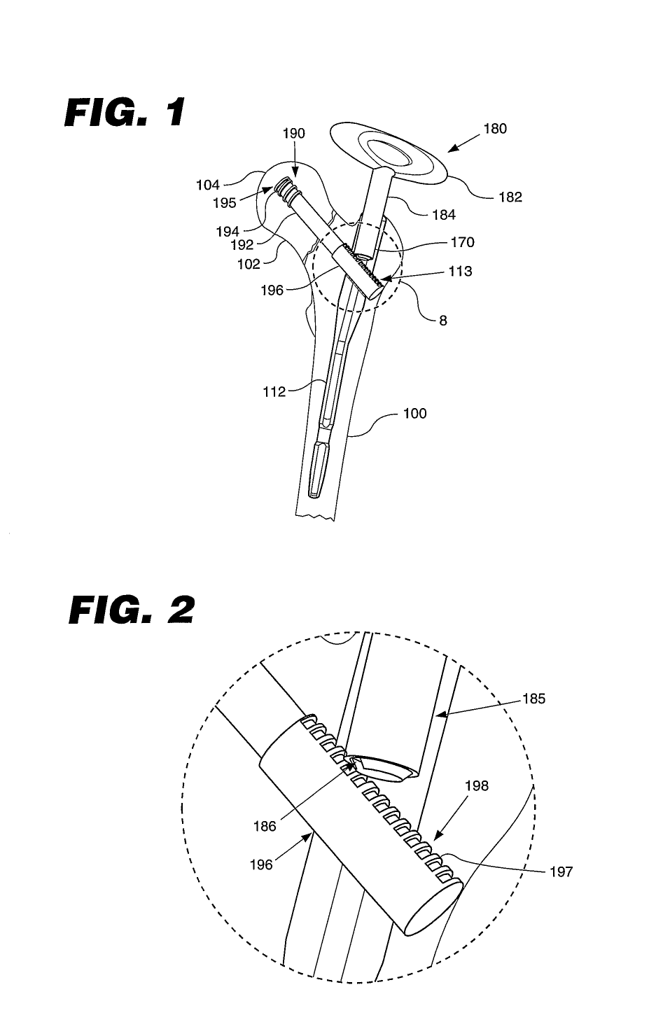

Referring to the accompanying drawings in which like reference numbers indicate like elements, FIGS. 1 and 2 illustrate a femur 100, a femoral neck 102, a femoral head 104, and a first instrument for reducing a fracture of a bone. In the depicted embodiment, the first instrument is used to reduce a fracture on a femoral neck of a femur. The first instrument includes an orthopaedic surgical implant 112, an implant member 190, and a driving member 180. In the embodiment depicted in FIG. 1, the orthopaedic surgical implant 112 is an intramedullary nail. The intramedullary nail 112 has a longitudinally extending bore 170 and a transverse hole 113. The implant member 190 is associated with or connected to the transverse hole 113. In the embodiment depicted in FIG. 1, the implant member 190 is slidingly engaged with the transverse hole 113. In some embodiments, the implant member 190 is cannulated to allow delivery of a material to the bone. The material may be a bone cement, a biologic, or a medicament. As an example only, the bone cement may be an adhesive bone cement of the kind disclosed in PCT Publication No. WO/2004/028576 or U.S. Patent Application Publication No. 2006/0096504, the contents of each publication is herein incorporated by reference in its entirety. Thus, the bone cement may include compositions of a calcium component and a liquid component, wherein the liquid component includes pyrophosphate ions with either or both of orthophosphate ions and water. Typically, the calcium component may be one or more of β-tricalcium phosphate (β-TCP), α-tricalcium phosphate (α-TCP), tetracalcium phosphate (TTCP), dicalcium phosphate anhydrous (DCPA), dicalcium phosphate dihydrate (DCPD), hydroxyapatite (HA) or calcium oxide (CaO). Suitable sources of pyrophosphate ions include pyrophosphoric acid or non-toxic, soluble pyrophosphate salts, which are aptly sodium salts and suitably tetrasodium pyrophosphate, disodium dihydrogen pyrophosphate and the like. Suitable sources of orthophosphate ions include orthophosphoric acid or non-toxic, soluble orthophosphate salts, which are aptly sodium salts.

The implant member 190 includes a shank 192. The shank 192 has a bone engagement portion 195 at a first end portion 194 and a driven portion 197 at a second end portion 196. In some embodiments, the bone engagement portion 195 is threaded. The driving member 180 is in driving engagement with the implant member 190. The driving member 180 has a shaft 184 with a driving end 186 at a third end portion 185. The shaft 184 is sized to fit within the longitudinally extending bore 170. The driving end 186, also known as a driving arm, selectively engages the driven portion 197 to move the implant member 190 when the driving member 180 is rotated. In the embodiment depicted in FIG. 1, the driven portion 197 includes serrations 198, and the driving arm 186 engages at least one of the serrations 198 to move the implant member 190. In some embodiments, the driving member 180 includes a handle 182. As the handle 182 and the driving end 186 are rotated, the implant member 190 is deployed toward or away from the head 104. The handle 182 may be rotated so that the fracture can be reduced. The driving end 186 can also be used to remove implant member 190 by rotating the handle 182 in the opposite direction. The driving member 180 may be made of a durable metallic, polymer, composite, plastic, or some combination thereof. Implant member 190 may be made of a polymer, composite, metal, biological, biodegradable, bioresorbable, plastic or some combination thereof.

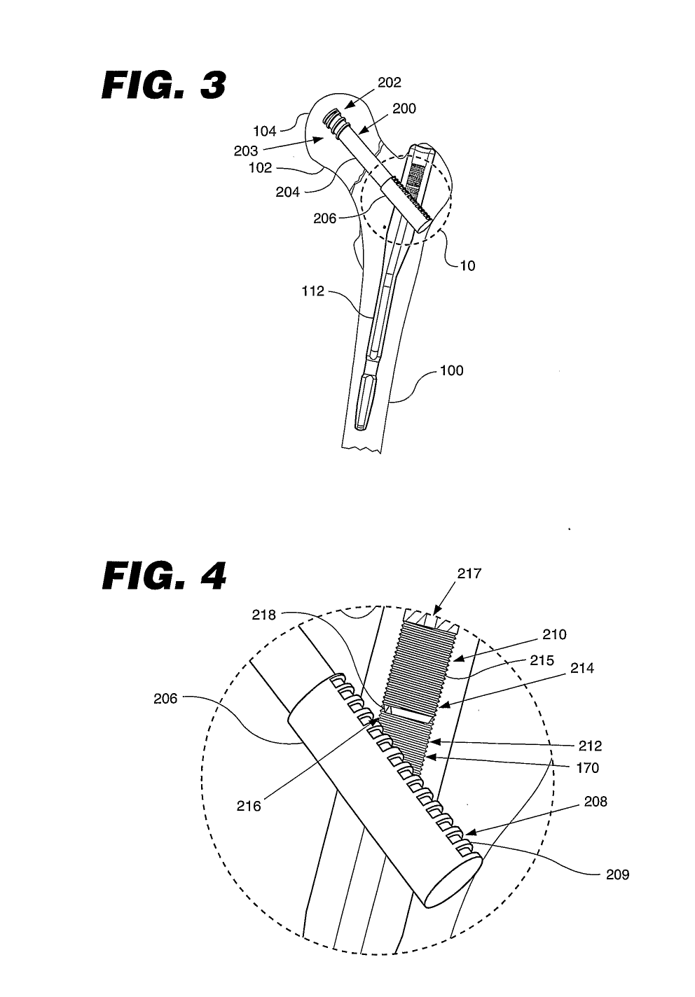

FIGS. 3 and 4 illustrate a second instrument for reduction of a bone fracture. The second instrument is similar to the first instrument. The second instrument includes the orthopaedic surgical implant 112, an implant member 200, and a driving member 210. In the embodiment depicted in FIG. 3, the orthopaedic surgical implant 112 is an intramedullary nail. The intramedullary nail 112 includes the longitudinally extending bore 170 and the transverse hole 113. The implant member 200 is connected to the transverse hole 113. In the embodiment depicted in FIG. 3, the implant member 200 is slidingly engaged with the transverse hole 113. In some embodiments, the implant member 200 is cannulated to allow delivery of a material to the bone. The implant member 200 includes a shank 204. The shank 204 has a bone engagement portion 203 at a first end portion 202 and a driven portion 208 at a second end portion 206. In some embodiments, the bone engagement portion 203 is threaded.

The driving member 210 is in driving engagement with the implant member 200. In some embodiments, the driving member 210 is a part of the implant and remains in the intramedullary nail 112 after compression is achieved. As examples, the driving member 210 may be a set screw, nail cap, a button, a gear or other similar device. The driving member 210 has a shaft 214 with a driving end 216 at a third end portion 218. The shaft 214 is sized to fit within the longitudinally extending bore 170. In the embodiment depicted in FIG. 4, the longitudinally extending bore 170 includes threads 212, and the shaft 214 has complementary threads 215 that mate with threads 212. The driving end 216, also known as a driving arm, selectively engages the driven portion 208 to move the implant member 200 when the driving member 210 is rotated. In the embodiment depicted in FIG. 4, the driven portion 208 includes serrations 209, and the driving arm 216 engages at least one of the serrations 209 to move the implant member 200. In some embodiments, the driving member 210 includes a driving feature 217, such as a hex adapted to receive a driver. For example, a removable driver (not shown) may be used to engage the driving feature 217 to rotate the driving member 210. As the driving feature 217 and the driving end 216 are rotated, the implant member 200 is deployed toward or away from the head 104. The driving end 216 can also be used to remove implant member 200 by rotating the driving feature 217 in the opposite direction. The driving member 210 may be made of a durable metallic, polymer, composite, plastic, or some combination thereof. Implant member 200 may be made of a polymer, composite, metal, biological, biodegradable, bioresorbable, plastic or some combination thereof.

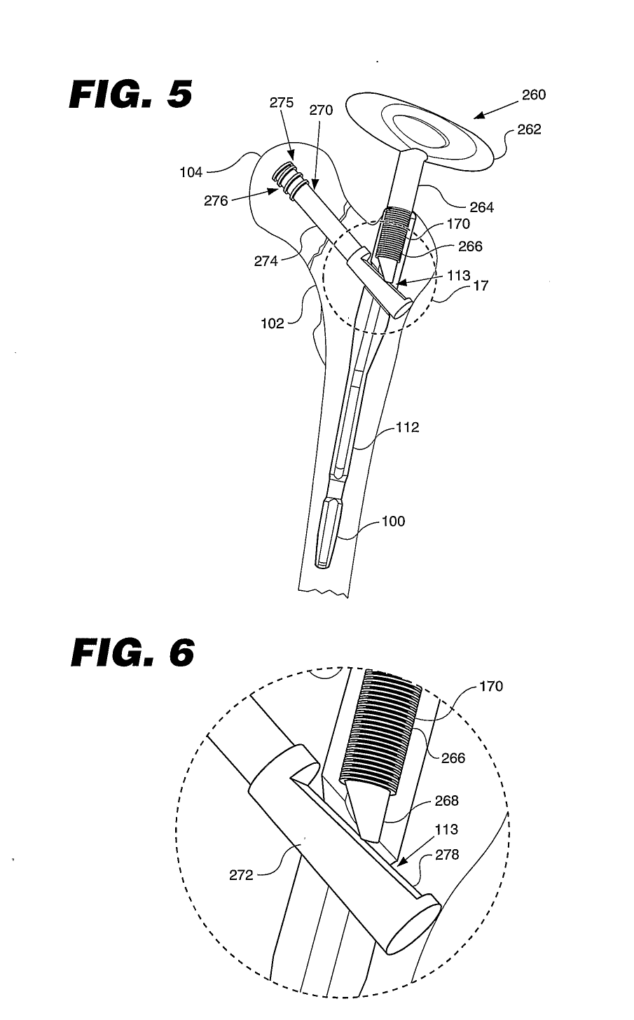

FIGS. 5 and 6 illustrate a third instrument for reducing a fracture of a bone. In the depicted embodiment, the third instrument is used to reduce a fracture on a femoral neck of a femur. The third instrument includes the orthopaedic surgical implant 112, an implant member 270, and a driving member 260. In the embodiment depicted in FIG. 5, the orthopaedic surgical implant 112 is an intramedullary nail. The intramedullary nail 112 has a longitudinally extending bore 170 and a transverse hole 113. The implant member 270 is connected to the transverse hole 113. In the embodiment depicted in FIGS. 5 and 6, the implant member 270 is slidingly engaged with the transverse hole 113. In some embodiments, the implant member 270 is cannulated to allow delivery of a material to the bone. The implant member 270 includes a shank 274. The shank 274 has a bone engagement portion 275 at a first end portion 276 and a driven portion 278 at a second end portion 272. In some embodiments, the bone engagement portion 275 is threaded.

The driving member 260 is in driving engagement with the implant member 270. The driving member 260 has a shaft 264 with a driving end 268 at a third end portion 266. The shaft 264 is sized to fit within the longitudinally extending bore 170. In the embodiment depicted in FIGS. 5 and 6, the third end portion 266 is threadingly engaged with the longitudinally extending bore 170. The driving end 268, also known as a driving arm, selectively engages the driven portion 278 to move the implant member 270 when the driving member 260 is rotated. In the embodiment depicted in FIG. 6, the driven portion 278 is an angled wedge, and the driving arm 268 engages the angled wedge to move the implant member 270. In some embodiments, the driving member 260 includes a handle 262. As the handle 262 and the driving end 268 are rotated, the implant member 270 is deployed toward or away from the head 104. The handle 262 may be rotated so that the fracture can be reduced. The driving end 268 can also be used to remove implant member 270 by rotating the handle 262 in the opposite direction. The driving member 260 may be made of a durable metallic, polymer, composite, plastic, or some combination thereof. Implant member 270 may be made of a polymer, composite, metal, biological, biodegradable, bioresorbable, plastic, or some combination thereof.

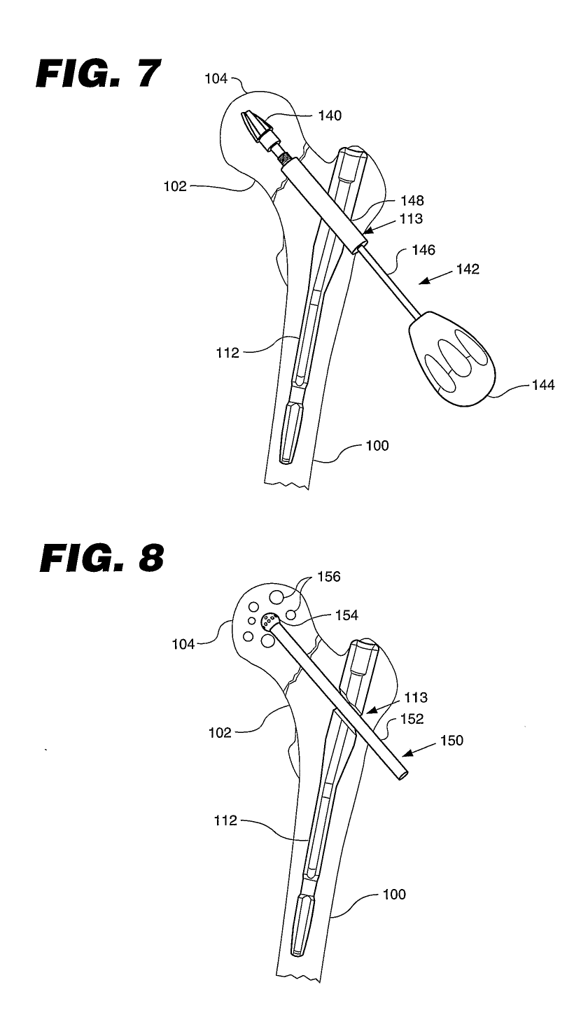

FIG. 7 illustrates a fourth instrument 142 for reducing a fracture. The fourth instrument 142 includes a tip member 140, a first handle 144, a first shaft 146, and a shank member 148. As examples, the tip member 140 may be a tap, a helical blade, or a drill bit. The shank member 148 is connected to the first shaft 146. In some embodiments, the shank member 148 is implantable. The shank member 148 can serve as an implantable fixation member to maintain the reduction of the fracture in the proximal femur. In some embodiments, the shank member 148 and the first shaft 146 are one-piece. In other embodiments, the first shaft member is connected to the tip member 140, and the first shaft 146 slides within the shank member 148. The shank member 148 is inserted through the orthopaedic surgical implant 112, such as an intramedullary nail. For example, the intramedullary nail 112 may include the transverse hole 113, and the shank member 148 may extend through the transverse hole 113. The tip member 140 may be attached to the shank member 148 through the use of a threaded connection. Alternatively, the tip member 140 may be attached to the shank member 148 through a press-fit, snap-fit, or through the use of a quick release mechanism. In this embodiment, the tip member 140 is inserted into the head 104 of the femur 100. The first handle 144 is used to position the tip member 140 into the desired location. The shank member 148 can slide and rotate relative to the transverse hole 113 to allow movement of the tip member 140. Alternatively, the first shaft 146 can slide and rotate relative to the shank member 148 to allow movement of the tip member 140. Once positioned, the tip member 140 can be manipulated to achieve reduction of the fracture.

FIG. 8 illustrates a fifth instrument 150, also known as a material applicator or a delivery device. The delivery device 150 is used to insert a material 156 into the fracture area of the bone. In the depicted embodiment, the delivery device 150 is used to insert the material 156 into the femoral head and/or neck. As examples, the material 156 may be bone cement, a biologic, or a medicament. The delivery device 150 includes a duct 152. The duct 152 is cannulated to allow for the delivery of the material 156 through the lumen of the device. An end 154 of the delivery device 150 is fenestrated or porous and enables material from the lumen to be positioned within the desired position in the bone. The delivery device 150 may be made of a polymer, composite, metal, biological, biodegradable, bioresorbable, plastic, or some combination thereof. In the embodiment depicted in FIG. 8, the duct 152 is inserted through the aperture 113. Once material 156 is deployed into and around the fracture site, it may become at least partially solidified. Thereafter, the fracture can be reduced by manipulating the at least partially solidified area. A more permanent implant then may be inserted through the at least partially set material 156 to permanently hold the fracture in a reduced configuration.

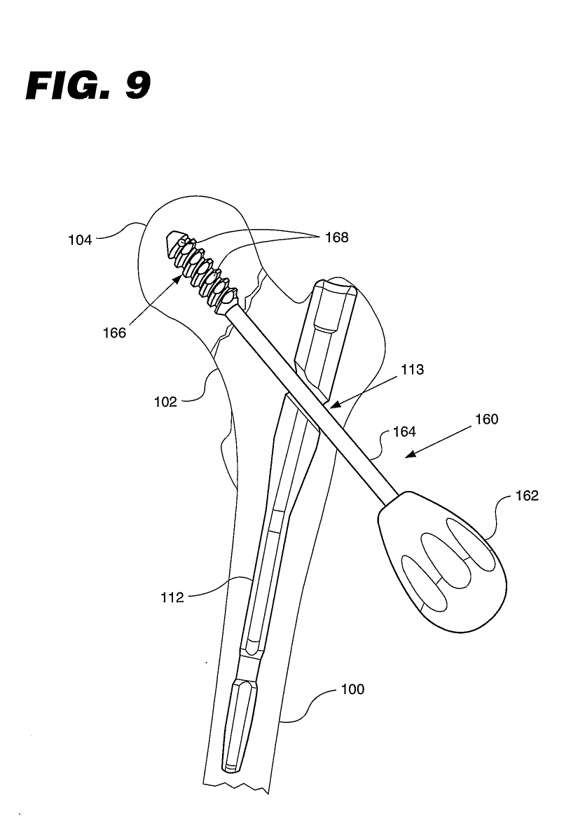

FIG. 9 illustrates an alternative embodiment of the fifth instrument. This embodiment includes a driving member 160, which may be used to reduce a fracture. The driving member 160 includes a second handle 162, a second shaft 164, and an end tap member 166. The tap member 166 can be inserted into the femoral head 104 and manipulated to achieve reduction. In the embodiment depicted in FIG. 9, the second shaft 164 is inserted through the aperture 113. The end tap member 166 may include a helical thread that runs along a portion or the entire length of the tap. The tap member 166 may be made of a polymer, composite, metal, biological, biodegradable, bioresorbable, plastic or some combination thereof.

In some embodiments, the tap member 166 includes holes or porous openings 168. The openings 168 are used to deploy materials, such as bone cement, a biologic, or a medicament. Once materials are deployed into and around the fracture site, the materials at least partially solidify and are capable of achieving fracture reduction. A more permanent implant may then be inserted through the set material to permanently hold the fracture in a reduced configuration.

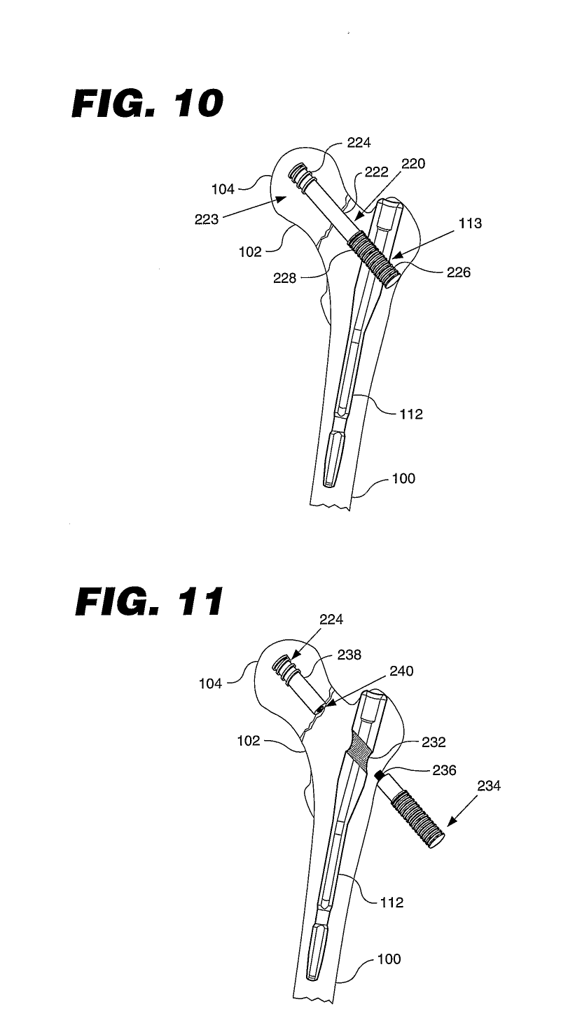

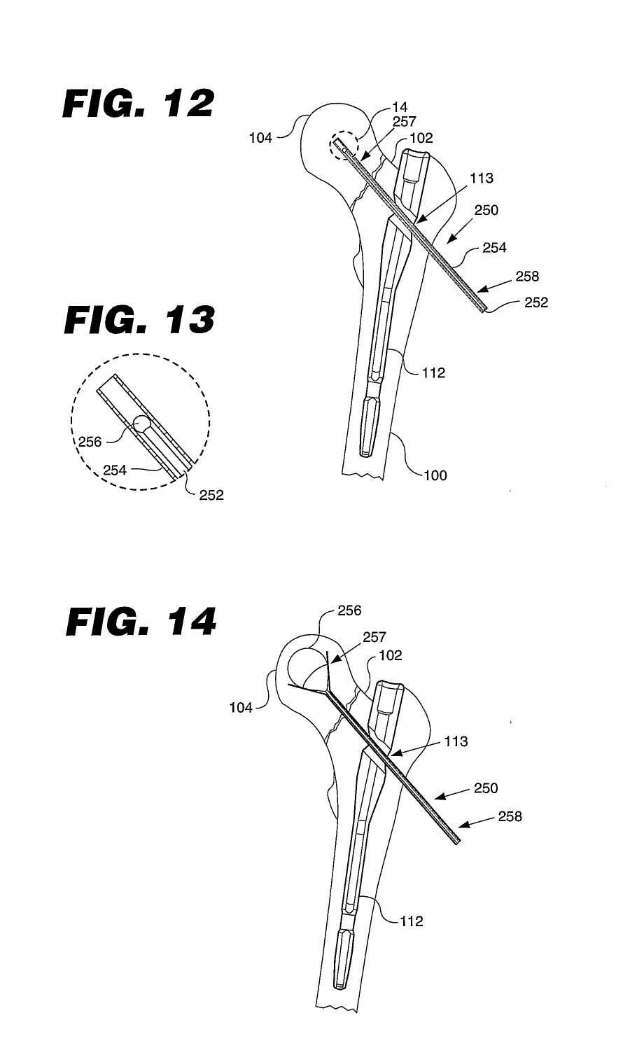

FIGS. 10 and 11 illustrate a sixth instrument that enables the reduction of a bone fracture. The sixth instrument includes the orthopaedic surgical implant 112 and an implant member 220. In the depicted embodiment, the orthopaedic surgical implant 112 is an intramedullary nail. The intramedullary nail 112 includes the transverse hole 113. In the embodiment depicted in FIG. 12, the transverse hole 113 includes threads 232. The implant member 220 is connected to the transverse hole 113. In the embodiments depicted in FIGS. 10 and 11, the implant member 220 is threadingly engaged with the transverse hole 113. The implant member 220 includes a main shaft 222. In some embodiments, the main shaft 222 is cannulated to allow delivery of a material to the bone. The main shaft 222 has a first end portion 224 and a second end portion 226. The first end portion 224 includes a bone engagement portion 223. In some embodiments, the bone engagement portion 223 is threaded. The second end portion 226 includes threads 228 that are complementary to and mate with the threads 232 of the transverse hole 113. After the bone engagement portion 223 is inserted into the bone the two threaded portions can be manipulated to achieve and maintain fracture reduction. The implant member 220 may be made of a polymer, composite, metal, biological, biodegradable, bioresorbable, plastic, or some combination thereof.

The sixth instrument may be a one part device (as best seen in FIG. 10) or a two part device (as best seen in FIG. 11). Accordingly, FIG. 11 illustrates the implant member 220 having a bone engagement member 238 and a driven member 234. The driven member 234 is removably attached to the bone engagement member 238. In the depicted embodiment, the driven member 234 has a threaded tip 236 which is received by a threaded hole 240 of the bone engagement member 238, but those of ordinary skill in the art would understand that other techniques may be used to accomplish this connection.

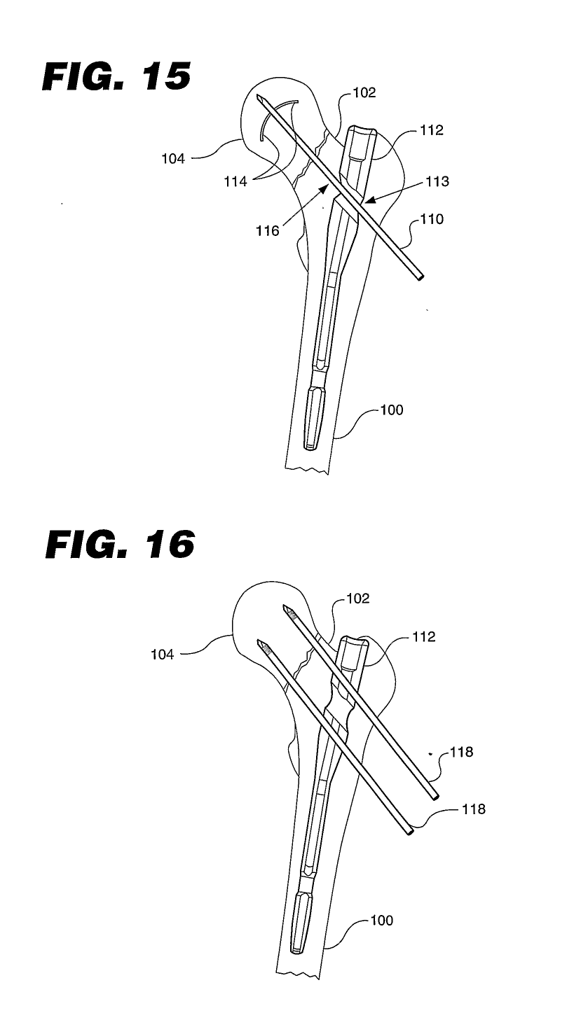

FIGS. 12, 13, and 14 illustrate a seventh instrument for reduction of a bone fracture. The seventh instrument includes the orthopaedic surgical implant 112 and an implant member 250. In the depicted embodiment, the orthopaedic surgical implant is an intramedullary nail. The intramedullary nail 112 includes the transverse hole 113. The implant member 250 is connected to the transverse hole 113. In the embodiments depicted in FIGS. 13 and 15, the implant member 250 is slidingly engaged with the transverse hole 113. The implant member 250 includes a main shaft 254. The main shaft 254 is cannulated to allow delivery of a support member 252 and an expanding element 256. The main shaft 254 has a first end portion 257 and a second end portion 258. The expanding element 256 may be a balloon or other inflatable device. In FIG. 13, the embodiment is shown in its reduced state for insertion into the desired location of the bone. The main shaft 254 and the support member 252 are deployed into the area of a fracture, such as the femoral head. Once deployed into the desired location, the expanding element 256 is expanded or inflated (as best seen in FIG. 14) so that its expansion causes a portion of the main shaft 254 to also expand. The main shaft 254 may be a stent-like structure that is used for achieving reduction of the fracture. The seventh instrument also can be used to expand and compress portions of bone. The main shaft 254 and the expanding element 256 are capable of achieving a rigid fixation that can be manipulated to achieve an anatomical reduction of a fracture. The main shaft 254 and/or the support member 252 may be made of shape memory metal or nonmetal, temperature memory metal or nonmetal, biodegradable or bioresorbable materials, polymers, composite materials, biologics, plastic materials, or some combination thereof. The main shaft 254 and/or the support member 252 may include a coating of radiopaque material to help in imaging or may comprises a coating of drugs, BMP, or other material to enhance the healing of the fractured area.

Another embodiment of the seventh instrument might include an oversized guide tip end that when retracted away from the fracture site would mechanically force out the main shaft. In this embodiment, the expanding element 256 is rigid and not inflatable. The expanding element 256 is oversized and capable of spreading the first end portion 257 through mechanical force. In this case, the first end portion 257 may have lengthwise slits or cuts to aid in its expansion. Initially, the expanding element 256 is located distally outside of the main shaft 254. In some instances, the support member 252 is assembled to the main shaft 254. The support member 252 and the main shaft 254 are inserted into the femoral head 104. The support member 252 is moved relative to the main shaft 254, thereby spreading the first end portion 257 through mechanical force as the expanding element 256 presses against the first end portion. The main shaft 254 could then be manipulated to achieve appropriate reduction and/or could be used to hold the fracture in place.

FIG. 15 illustrates an eighth instrument 110 for reduction of a bone fracture. The eighth instrument 110 is used in conjunction with the orthopaedic surgical implant 112. In the embodiment depicted in FIG. 15, the orthopaedic surgical implant 112 is an intramedullary nail, and the intramedullary nail 112 has been inserted into an intramedullary canal (not shown) prior to insertion of the first instrument 110. The intramedullary nail 112 includes an aperture or transverse hole 113. The aperture 113 is transverse to the longitudinal axis of the intramedullary nail 112. The eighth instrument 110 is inserted through the aperture 113, through the femoral neck 102, and toward the femoral head 104. When correctly positioned within the femoral head 104, deployable members 114 are released from the body 116. Deployable members 114 extend radially away from the first body 116 and are able to maintain a rigid fixation within the femoral head 104. This enables the surgeon to reduce the fracture by manipulating or pulling on the body 116 while the deployable members 114 maintain their positioning in the femoral head 104. The deployable members 114 may include teeth, talons, fins, barbs, pins, or wires. The eighth instrument 110 may be made of a durable metallic, polymer, composite, plastic, or some combination thereof. Alternatively, the first instrument 110 could be made of a material that is bioresorbable.

FIG. 16 illustrates a ninth instrument that includes a plurality of fixation members 118 that are implanted into the head 104 of the femur 100. As examples, the fixation members 118 may be guide pins or half pins. The fixation members 118 are inserted past the orthopaedic surgical implant 112, through the neck 102, and into the head 104. In the depicted embodiment, the orthopaedic surgical implant 112 is an intramedullary nail. For example, the fixation members 118 may be inserted on either side of the intramedullary nail 112 or through apertures within it. Once inserted into the patient's bone, the fixation members 118 can be utilized to achieve reduction of the fracture by manipulating the fixation elements 118 into a desirable orientation. The fixation members 118 may be held while an additional device is used to insert an implant into the head 104 of the femur 100 to maintain the reduction on a more permanent basis.

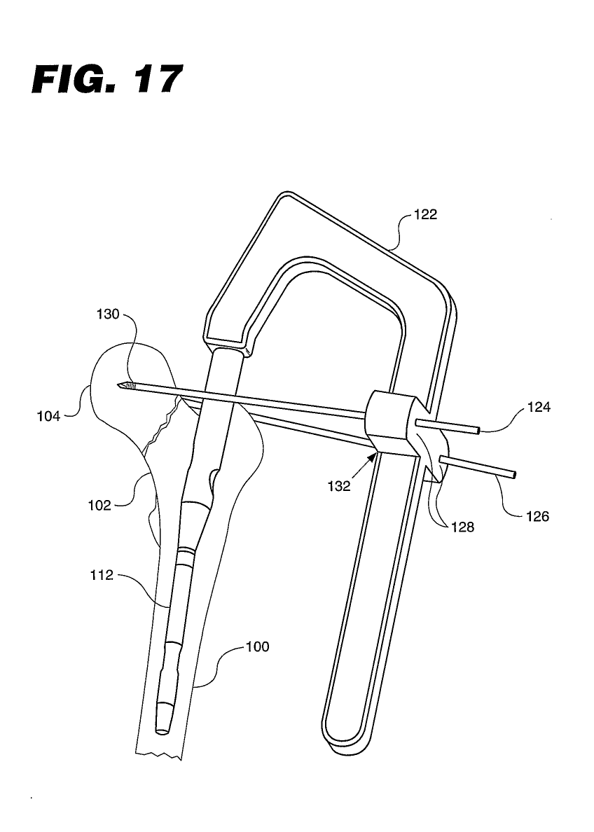

FIG. 17 illustrates a tenth instrument that includes a guide 122 and fixation elements 124, 126. The guide 122 enables the reduction of a fracture. The guide 122 connects to the end of the orthopaedic surgical implant 112. For example, the guide 122 may connect to the orthopaedic surgical implant 112 in the way a drill guide connects to an intramedullary nail. Fixation elements 124, 126 connect to the guide 122 and are inserted into the head 104. The fixation elements 124, 126 are inserted past the orthopaedic surgical implant 112 and into the head 104. For example, the fixation elements 124, 126 may be inserted on either side of the orthopaedic surgical implant 112. The fixation elements 124, 126 may be threaded or sharp on one end 130 to enable them to cut through bone and maintain a rigid fixation within the bone. The guide 122 includes a holding member 132. The holding member 132 includes holes 128. The holes 128 receive the fixation elements 124, 126. The holding member 132 is used to hold and maintain the fixation elements 124, 126 in a fixed position. Once the fixation elements 124, 126 are inserted into the desired area, they can be manipulated to reduce the fracture. Therefore, if the fixation elements 124, 126 are used to compress a fracture of the femoral neck, they can be used to maintain that compression while a permanent implant is inserted to permanently achieve fixation, reduction or compression of the fracture.

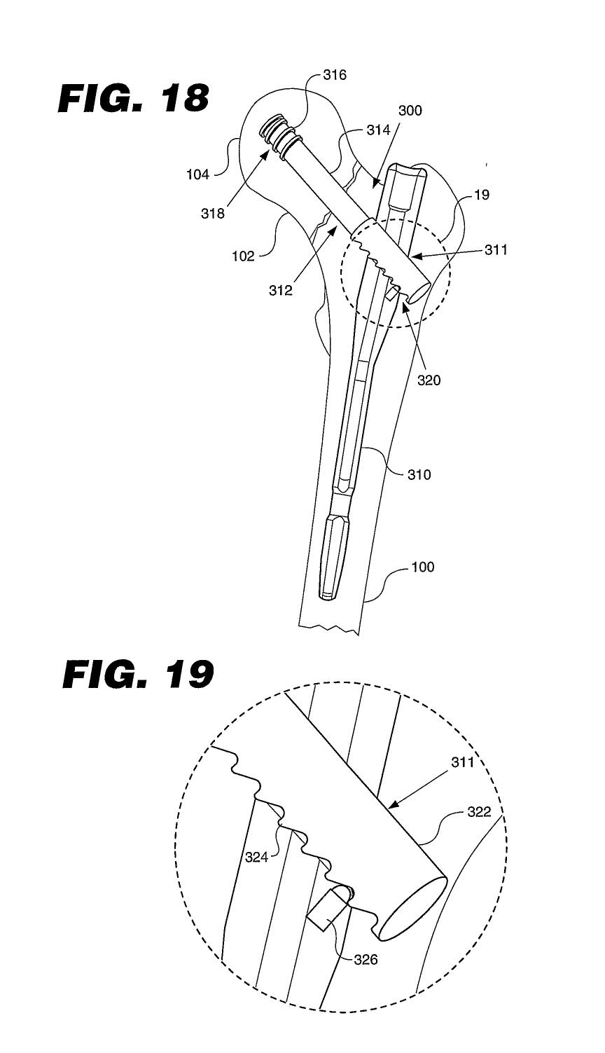

FIGS. 18 and 19 illustrate a sliding compression orthopaedic implant 300 and the femur 100. As an example, the implant 300 may be applied to a fracture of the femoral neck 102. The implant 300 maintains the reduction of the fracture but allows for dynamic loading to aid in fracture healing. The implant 300 includes a first implant member 310 and a second implant member 312. In the embodiment depicted in FIG. 18, the first implant member 310 is an intramedullary nail. The intramedullary nail 310 has a transverse hole 311, and the second implant member 312 is connected to the transverse hole 311. In the depicted embodiments, the second implant member 312 slidingly engages the transverse hole 311. The second implant member 312 has a shank 314, and the shank 314 has a bone engagement portion 316 at a first end portion 318 and a sliding compression member 320 at a second end portion 322. In some embodiments, the bone engagement portion 316 is threaded. As best seen in FIG. 19, the sliding compression member 320 is a ratchet mechanism that includes serrations 324 located on the second end portion 322 and a pin 326. The pin 326 is depressable but biased to engage one of the serrations 324. The ratchet mechanism allows the implant member 312 to move only in one direction when a load is applied along its length.

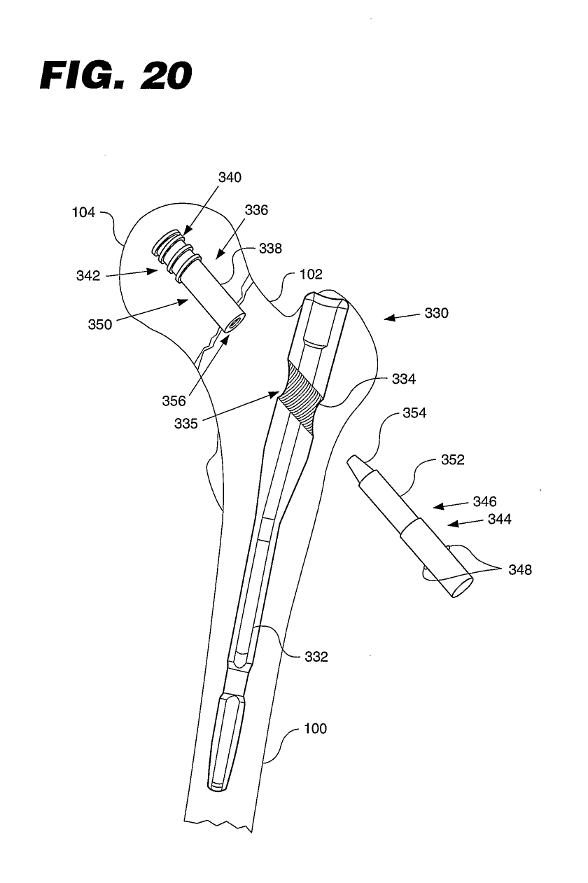

FIG. 20 illustrates a second embodiment 330 of the sliding compression orthopaedic implant and the femur 100. The implant 330 maintains the reduction of the fracture but allows for dynamic loading to aid in fracture healing. The implant 330 includes a first implant member 332 and a second implant member 336. In the embodiment depicted in FIG. 20, the first implant member 332 is an intramedullary nail. The intramedullary nail 332 has a transverse hole 334, and the second implant member 336 is connected to the transverse hole 334. In the depicted embodiments, the transverse hole 334 includes threads 335. The second implant member 336 has a shank 338, and the shank 338 has a bone engagement portion 340 at a first end portion 342 and a sliding compression member 344 at a second end portion 346. In some embodiments, the bone engagement portion 340 is threaded. As best seen in FIG. 19, the sliding compression member 344 includes at least one expanding element 348 that is radially biased away from the shank 338 and engages the threads 335 of the transverse hole 334. In the embodiment depicted in FIG. 20, the second end portion 346 includes two expanding elements 348. The expanding elements 348 interact with the transverse hole 334 to maintain the second implant member 336 in a compression loading condition.

The second implant member 336 may be a one part device or a two part device. Accordingly, the second implant member 336 may have a bone engagement member 350 and a driven member 352. The driven member 352 is removably attached to the bone engagement member 350. In the depicted embodiment, the driven member 352 has a taper 354, such as a Morse taper, which is received by a tapered hole 356 of the bone engagement member 350, but those of ordinary skill in the art would understand that other techniques may be used to accomplish this connection.

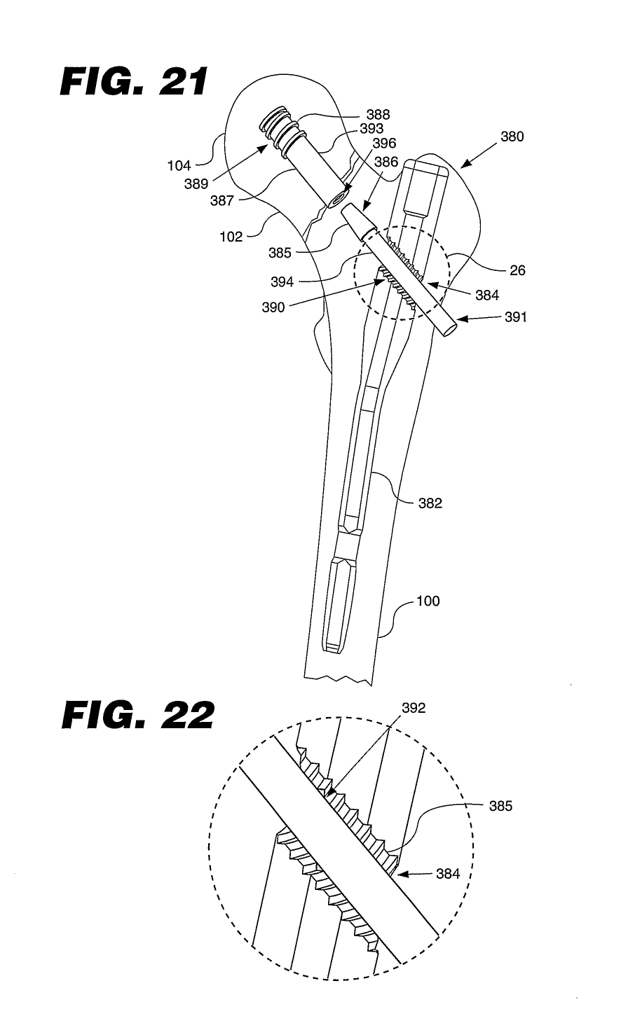

FIGS. 21 and 22 illustrate a third embodiment 380 of the sliding compression orthopaedic implant and the femur 100. The implant 380 maintains the reduction of the fracture but allows for dynamic loading to aid in fracture healing. The implant 380 includes a first implant member 382 and a second implant member 386. In the embodiment depicted in FIG. 21, the first implant member 382 is an intramedullary nail. The intramedullary nail 382 has a transverse hole 384, and the second implant member 386 is connected to the transverse hole 384. In the depicted embodiments, the transverse hole 384 includes grooves 385. As examples, the grooves 385 may be circular or helical. Further, the grooves 385 may be machined or molded into the intramedullary nail 382.

The second implant member 386 has a shank 387, and the shank 387 has a bone engagement portion 388 at a first end portion 389 and a sliding compression member 390 at a second end portion 391. In some embodiments, the bone engagement portion 388 is threaded. As best seen in FIG. 22, the sliding compression member 390 includes at least one fin 392 that engages the grooves 385 of the transverse hole 384. In the embodiment depicted in FIG. 22, the second end portion 391 includes two fins 392. The fins 392 interact with the transverse hole 384 to maintain the second implant member 386 in a compression loading condition. As a load is applied to the second implant member 386, the fins 392 toggle or ratchet in the direction in which the load is applied, thus allowing compression. The fins 392 could also be modular elements, such as keys, that are inserted after the second end portion 391 is inserted into the hole 384.

The second implant member 386 may be a one part device or a two part device. Accordingly, the second implant member 386 may have a bone engagement member 393 and a driven member 394. The driven member 394 is removably attached to the bone engagement member 393. In the depicted embodiment, the driven member 394 has a taper 395, such as a Morse taper, which is received by a tapered hole 396 of the bone engagement member 393, but those of ordinary skill in the art would understand that other techniques may be used to accomplish this connection.

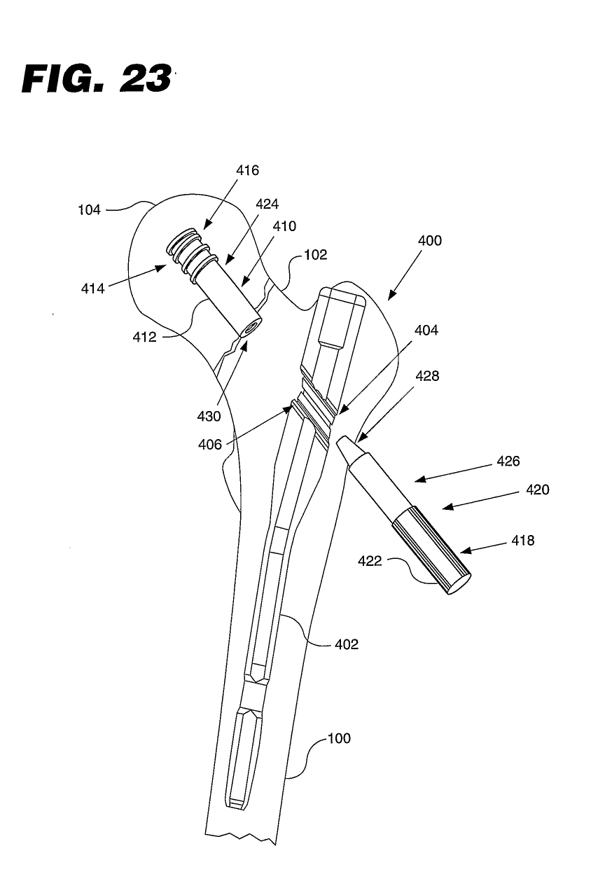

FIG. 23 illustrates a fourth embodiment 400 of the sliding compression orthopaedic implant and the femur 100. The implant 400 maintains the reduction of the fracture but allows for dynamic loading to aid in fracture healing. The implant 400 includes a first implant member 402 and a second implant member 410. In the embodiment depicted in FIG. 23, the first implant member 402 is an intramedullary nail. The intramedullary nail 402 has a transverse hole 404, and the second implant member 410 is connected to the transverse hole 404. In the depicted embodiments, the transverse hole 404 includes transverse grooves 406.

The second implant member 410 has a shank 412, and the shank 412 has a bone engagement portion 414 at a first end portion 416 and a sliding compression member 418 at a second end portion 420. In some embodiments, the bone engagement portion 414 is threaded, tapped, tapered or fluted to enable it to be inserted into the bone. As best seen in FIG. 23, the sliding compression member 418 includes at least one tongue 422 that is sized to fit within one of the grooves 406 of the transverse hole 404. In the embodiment depicted in FIG. 23, the second end portion 420 has a plurality of tongues 422 adapted to mate with the grooves 406. In an alternative embodiment, the tongues 422 could be formed on the hole 404 and the grooves 406 could be formed on the second end portion 420. The tongue and groove combination prevents rotation but still enables sliding compression.

The implant member 410 may be a one part device or a two part device. Accordingly, the second implant member 410 may have a bone engagement member 424 and a driven member 426. The driven member 426 is removably attached to the bone engagement member 424. In the depicted embodiment, the driven member 426 has a taper 428, such as a Morse taper, which is received by a tapered hole 430 of the bone engagement member 424, but those of ordinary skill in the art would understand that other techniques may be used to accomplish this connection.

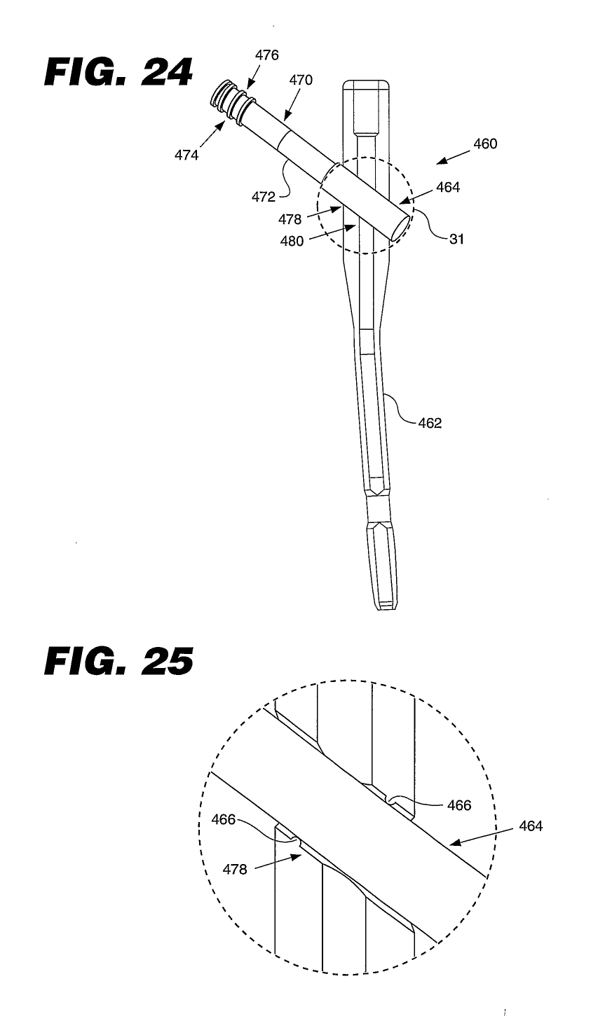

FIGS. 24 and 25 illustrate a fifth embodiment 460 of the sliding compression orthopaedic implant. The implant 460 includes a first implant member 462 and a second implant member 470. In the embodiment depicted in FIG. 24, the first implant member 462 is an intramedullary nail. The intramedullary nail 462 has a transverse hole 464, and the second implant member 470 is connected to the transverse hole 464. The transverse hole 464 includes at least one bearing 466, such as a roller ball bearing. In the depicted embodiments, the transverse hole 464 has two bearings 466.

The second implant member 470 has a shank 472, and the shank 472 has a bone engagement portion 474 at a first end portion 476 and a sliding compression member 478 at a second end portion 480. In some embodiments, the bone engagement portion 474 is threaded, tapped, tapered or fluted to enable it to be inserted into the bone. As best seen in FIG. 31, the sliding compression member 478 engages or rides on the bearings 466.

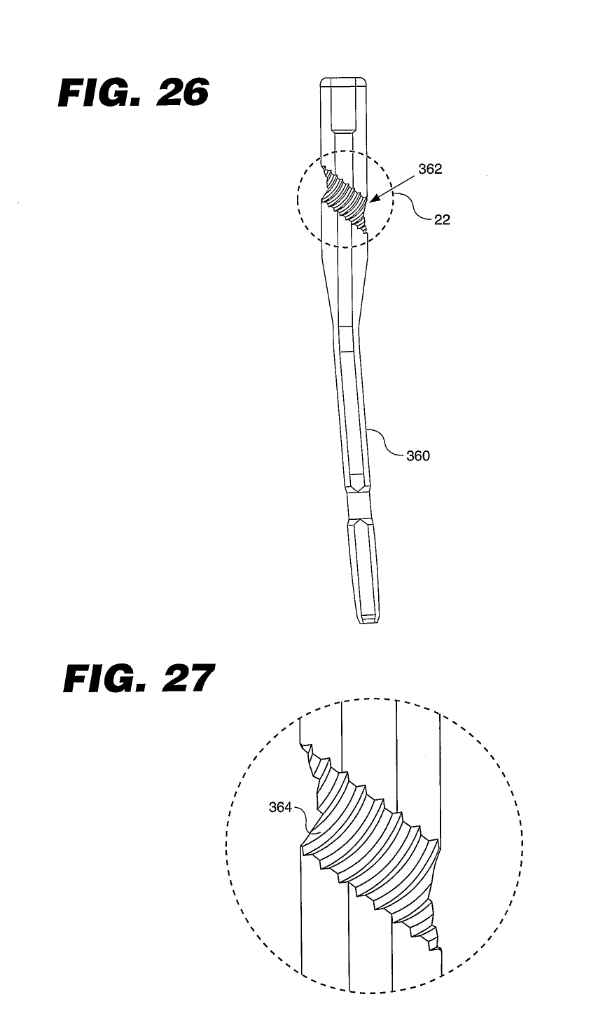

FIGS. 26, 27, 28, and 29 illustrate alternate embodiments of the intramedullary nail 360, 370. Each intramedullary nail 360, 370 has a hole 362, 372 that transverses its longitudinal axis. Each transverse hole 362, 372 has a geometric variation. In the embodiment depicted in FIG. 26, the geometric variation includes one or more circular grooves 364. As examples, the circular grooves 364 may be machined or molded into the nail 360. In some embodiments, the circular grooves 364 may be filled with a polymer, metallic, composite, ceramic, or biologic material to enable sliding compression. In one particular embodiment, the circular grooves 364 may be filled with a material, such as ultra high molecular weight polyethylene (UHMWPE), to enable a fixation element, such as a implant member, lag screw, rod, pin, angled cross-nail, locking screw, or two-part screw, to compress in one direction when loaded by the patient. The intramedullary nail 360 may be used in any of the inventions or embodiments described herein.

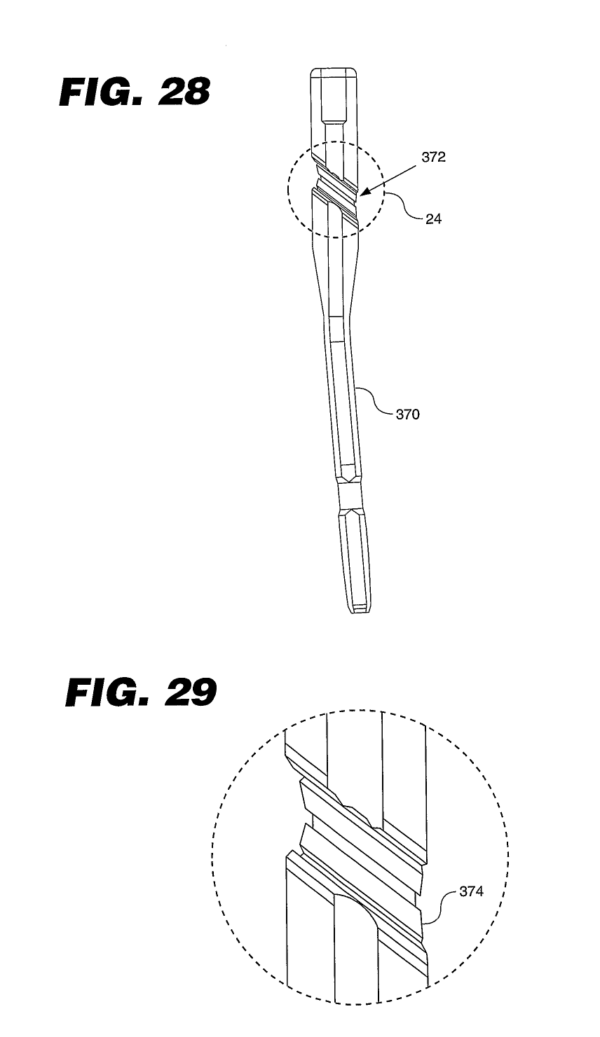

FIG. 28 is similar to that of FIG. 26. The intramedullary nail 370 has a geometric variation that includes transverse grooves 374. The transverse grooves 374 may be filled with body fluid to enable sliding compression. These grooves 374 may also contain a polymer, metallic, composite, ceramic, biologic, or other material that reduces friction and increases the efficiency of sliding compression. The intramedullary nail 370 may be used in any of the inventions or embodiments described herein.

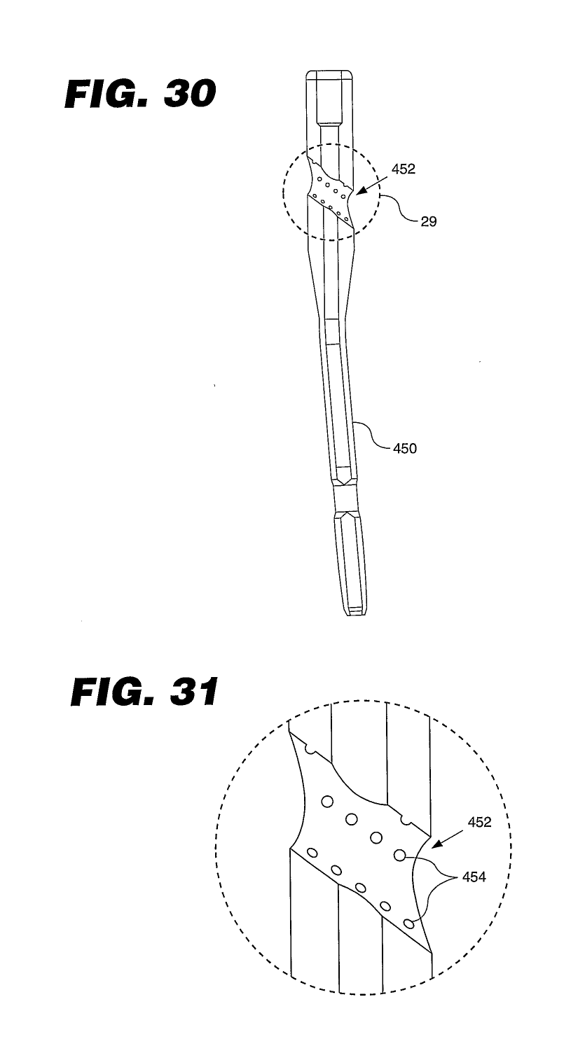

FIGS. 30 and 31 illustrate an alternate embodiment of the intramedullary nail 450. The intramedullary nail 450 has a hole 452 that transverses its longitudinal axis. The transverse hole 452 has a geometric variation. In the embodiment depicted in FIG. 28, the geometric variation includes one or more dimples 454. The dimples 454 may be similar in shape to dimples of a golf ball. As examples, the dimples 454 may be machined or molded into the nail 450. In some embodiments, the dimples 454 may be filled with a polymer, metallic, composite, ceramic, or biologic material to enable sliding compression. In one particular embodiment, the dimples 454 may be filled with a material, such as ultra high molecular weight polyethylene (UHMWPE), to enable a fixation element, such as a implant member, lag screw, rod, pin, angled cross-nail, locking screw, or two-part screw, to compress in one direction when loaded by the patient. The intramedullary nail 450 may be used in any of the inventions or embodiments described herein.

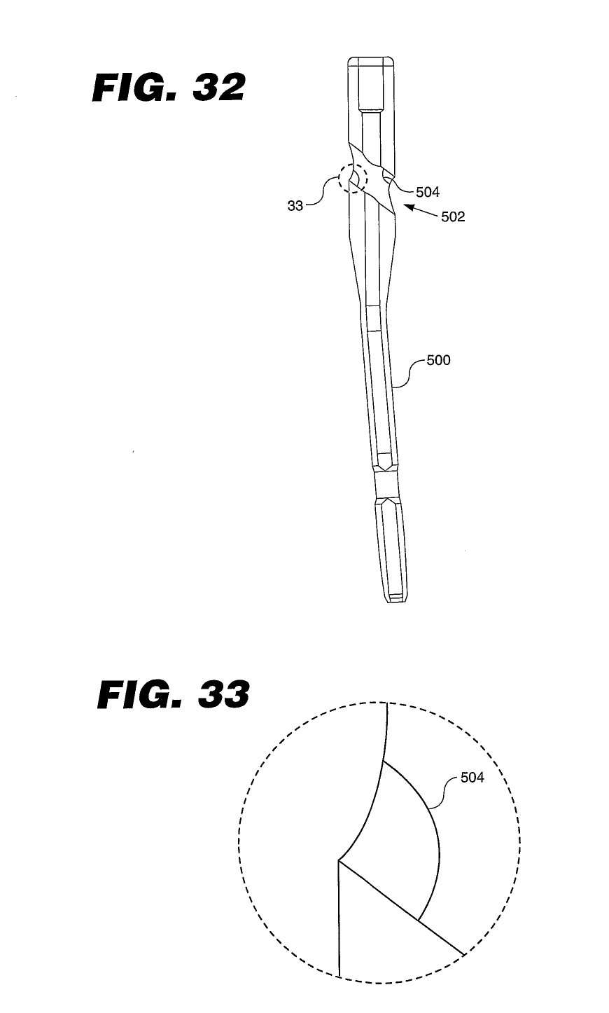

FIGS. 32 and 33 illustrate an alternate embodiment of the intramedullary nail 500. The intramedullary nail 500 has a hole 502 that transverses its longitudinal axis. The transverse hole 502 has a geometric variation. In the embodiment depicted in FIG. 33, the geometric variation includes one or more chamfers 504. As examples, the chamfers 504 may be machined or molded into the nail 500. The chamfers 504 allow a fixation element, such as an implant member, a lag screw, rod, pin, angled cross-nail, locking screw, two-part screw, to compress when loaded by the patient. The chamfered design increases the surface area with relation to the fixation element, thus reducing the stress. The chamfers 504 improve the compressibility of the device. The intramedullary nail 500 may be used in any of the inventions or embodiments described herein.

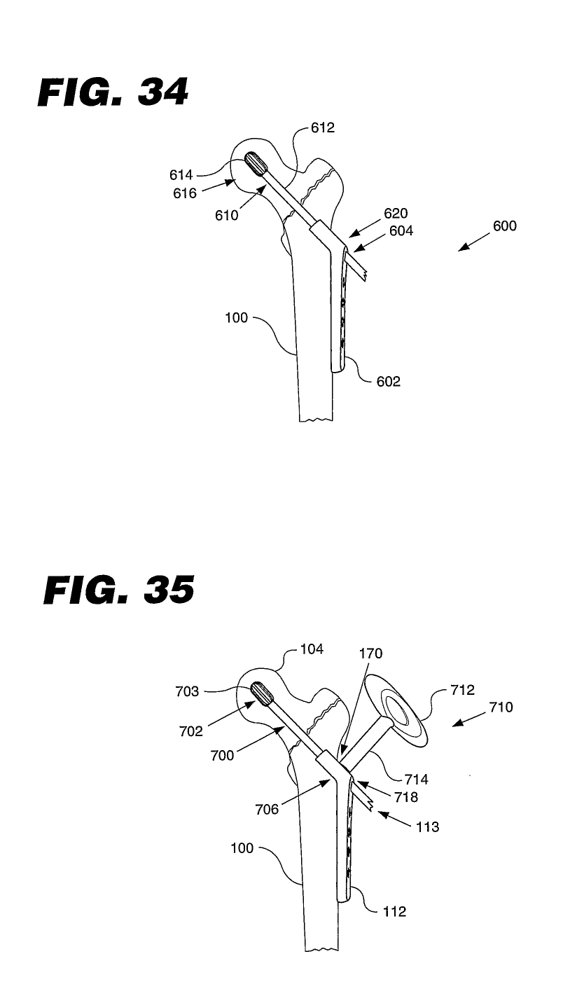

FIG. 34 illustrates a sixth embodiment 600 of the sliding compression orthopaedic implant and the femur 100. The implant 600 maintains the reduction of the fracture but allows for dynamic loading to aid in fracture healing. The implant 600 is very similar to the implant 400 illustrated in FIG. 23. The implant 600 includes a first implant member 602 and a second implant member 610. In the embodiment depicted in FIG. 34, the first implant member 602 is an extramedullary plate. The extramedullary plate 602 has a transverse hole 604, and the second implant member 610 is connected to the transverse hole 604. In the depicted embodiments, the transverse hole 604 includes transverse grooves (not shown).

The second implant member 610 has a shank 612, and the shank 612 has a bone engagement portion 614 at a first end portion 616 and a sliding compression member (not shown) at a second end portion 620. In some embodiments, the bone engagement portion 614 is threaded, tapped, tapered or fluted to enable it to be inserted into the bone. The sliding compression member includes at least one tongue (not shown) that is sized to fit within one of the grooves of the transverse hole 604. In an alternative embodiment, the tongues could be formed on the hole 604 and the grooves could be formed on the second end portion 620. The tongue and groove combination prevents rotation but still enables sliding compression.

Similar to the embodiments depicted in FIGS. 20, 21, and 23, the second implant member 610 may be a one part device or a two part device that includes a bone engagement member and a driven member.

FIG. 35 illustrates an eleventh instrument for reduction of a bone fracture. The eleventh instrument is similar to the first instrument. The eleventh instrument includes the orthopaedic surgical implant 112, an implant member 700, and a driving member 710. In the embodiment depicted in FIG. 35, the orthopaedic surgical implant 112 is an extramedullary plate. The extramedullary plate 112 includes the longitudinally extending bore 170 and the transverse hole 113. The implant member 700 is connected to the transverse hole 113. In the embodiment depicted in FIG. 35, the implant member 700 is slidingly engaged with the transverse hole 113. In some embodiments, the implant member 700 is cannulated to allow delivery of a material to the bone or to aid in the implant's installation. In the latter case, a guide wire (not shown) may be put in place prior to placing the implant member as to guide the implant member's trajectory. The implant member 700 includes a shank 704. The shank 704 has a bone engagement portion 703 at a first end portion 702 and a driven portion (not shown) at a second end portion 706. In some embodiments, the bone engagement portion 703 is threaded.

The driving member 710 is in driving engagement with the implant member 700. In some embodiments, the driving member 710 is a part of the implant and remains in the orthopaedic surgical implant 112 after compression is achieved, but in other embodiments the driving member 710 is removed after reduction and fixation. The driving member 710 has a shaft 714 with a driving end (not shown) at a third end portion 718. The shaft 714 is sized to fit within the longitudinally extending bore 170. The driving end, also known as a driving arm, selectively engages the driven portion to move the implant member 700 when the driving member 710 is rotated. In some embodiments, the driving member 710 includes a handle 712. As the handle 712 and the driving end are rotated, the implant member 700 is deployed toward or away from the head 104. The handle 712 may be rotated so that the fracture can be reduced. The driving end can also be used to remove implant member 700 by rotating the handle 712 in the opposite direction. The driving member 710 may be made of a durable metallic, polymer, composite, plastic, or some combination thereof. Implant member 700 may be made of a polymer, composite, metal, biological, biodegradable, bioresorbable, plastic, or some combination thereof.

The device disclosed herein provides the mechanical and biological advantages of intramedullary nailing along with the proven benefits of sliding compression in fracture healing. The devices disclosed herein provide a variety of options for treating fractures of the femoral neck. Several of the devices are more simplified and offer the advantages to the manufacturing process. Other devices offer improved mechanical properties over that of the prior art.

While the devices disclosed herein have been illustrated in use for the treatment of femoral fractures, those of ordinary skill in the art would understand that the concepts disclosed herein are equally applicable to the distal femur, proximal tibia, distal tibia, proximal fibula, distal fibula, proximal humerus, distal humerus, proximal radius, distal radius, proximal ulna, and distal ulna.

Further, while the orthopaedic surgical implant and the first implant member have only been illustrated as intramedullary nails and extramedullary plates, those of ordinary skill in the art would understand that these components could equally be an intramedullary plate.

In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated.

As various modifications could be made in the constructions and methods herein described and illustrated without departing from the scope of the invention, it is intended that all matter contained in the foregoing description or shown in the accompanying drawings shall be interpreted as illustrative rather than limiting. For example, while the instruments and techniques described herein are related to femoral fractures, those of ordinary skill in the art would understand that these instruments and techniques also could be used to treat bone fractures in other anatomical locations. Thus, the breadth and scope of the present invention should not be limited by any of the above-described exemplary embodiments, but should be defined only in accordance with the following claims appended hereto and their equivalents.

1. An instrument for reduction of a fracture of a bone, the instrument comprising:
a. an orthopaedic surgical implant, said orthopaedic surgical implant having a longitudinally extending bore and a transverse hole;
b. an implant member operatively connected to said transverse hole, said implant member having a shank, and said shank having a bone engagement portion at a first end portion and a driven portion at a second end portion;
c. a driving member in driving engagement with said implant member, said driving member having a shaft with a driving arm at a third end portion, said shaft sized to fit within said longitudinally extending bore, and said driving arm selectively engaged with said driven portion; and
d. wherein said driving member is rotated to move said implant member in order to reduce the fracture.

2. The instrument of claim 1, wherein said orthopaedic surgical implant is selected from the group consisting of an intramedullary nail and an extramedullary plate.

3. The instrument of claim 1, wherein said bone engagement portion is threaded.

4. The instrument of claim 1, wherein said implant member is cannulated to allow delivery of a material to the bone.

5. The instrument of claim 1, wherein said driven portion further comprises a plurality of serrations.

6. The instrument of claim 1, wherein said driven portion further comprises an angled wedge.

7. The instrument of claim 1, wherein said implant member is slidably engaged with said transverse hole.

8. The instrument of claim 1, wherein said implant member is threadingly engaged with said transverse hole.

9. The instrument of claim 1, wherein said driving member is threadingly engaged with said longitudinally extending bore.

10. The instrument of claim 1, wherein said driving member further comprises a driving feature.

11. The instrument of claim 1, wherein said driving member further comprises a handle connected to said shaft.

12. The instrument of claim 1, wherein said implant member is comprised of one-piece.

13. The instrument of claim 1, wherein said implant member is comprised of at least two-pieces.

14. The instrument of claim 1, wherein said shank of said implant member has a bone engagement member and a driven member.

15. A sliding compression orthopaedic implant, the implant comprising:
a. a first implant member, said first implant member having a transverse hole; and
b. a second implant member operatively connected to said transverse hole, said second implant member having a shank, and said shank having a bone engagement portion at a first end portion and a sliding compression member at a second end portion.

16. The implant of claim 15, wherein said first implant member is selected from the group consisting of an intramedullary nail and an extramedullary plate.

17. The implant of claim 15, wherein said sliding compression member comprises a ratcheting mechanism.

18. The implant of claim 15, wherein said sliding compression member comprises at least one groove.

19. The implant of claim 15, wherein said transverse hole includes at least one groove and said sliding compression member is at least one expanding element that engages said at least one groove.

20. The implant of claim 15, wherein said transverse hole includes at least one groove and said sliding compression member is at least one fin that engages said at least one groove.

21. The implant of claim 15, wherein said transverse hole includes at least one groove and said intramedullary nail includes a material placed within said at least one groove.

22. The implant of claim 15, wherein said transverse hole includes at least one dimple and said intramedullary nail includes a material placed within said at least one dimple.

23. The implant of claim 15, wherein said intramedullary nail further comprises at least one bearing mounted within said transverse hole.

24. The implant of claim 15, wherein said transverse hole further comprises at least one chamfer.

25. The implant of claim 15, wherein said bone engagement portion is threaded.

26. The implant of claim 15, wherein said implant member is cannulated to allow delivery of a material to the bone.

27. The implant of claim 15, wherein said implant member is slidably engaged with said transverse hole.

28. The implant of claim 15, wherein said implant member is threadingly engaged with said transverse hole.

29. The implant of claim 15, wherein said implant member is comprised of one-piece.

30. The implant of claim 15, wherein said implant member is comprised of at least two-pieces.

31. The implant of claim 15, wherein said shank of said implant member has a bone engagement member and a driven member.

32. The implant of claim 31, wherein said bone engagement member is connected to said driven member through the use of a morse taper.
